(19) United States
(12) Patent Application Publication
(10) Pub. No.: US 2011/0004167 A1
(43) Pub. Date: Jan. 6, 2011

(54) Totally implantable central venous access port

(76) Inventors: Po-Jen Ko, Taipei City (TW); Ching-Yang Wu, Linkou Township (TW); Yun-Hen Liu, Guishan Shiang (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/497,741

(22) Filed: Jul. 6, 2009

(51) Int. Cl. A61M 5/00

(52) U.S. Cl. 604/288.01

(57) ABSTRACT

An injection base for artificial blood vessels includes a body and a catheter. The body has an injection chamber, a strengthened injection top diaphragm and a strengthened injection side diaphragm. The catheter is connected to one side of the body, and the strengthened injection side diaphragm is at another side of the body according to the catheter. The body and the catheter are integrated as one member. The totally implantable central venous access port further has a metal wire, which is coated with a film, and a puncture sheath with a side opening in order to make a loop around the metal wire through the puncture sheath conveniently. For the cases of a small blood vessel and a large angle of a blood vessel, the totally implantable central venous access port is easily implanted into and reinforces the strength thereof.

### BACKGROUND OF THE INVENTION

1. Field of the Invention

The present invention generally relates to a totally implantable central venous access port, more particularly to an improved totally implantable central venous access port.

2. Description of the Prior Art

For cancer patients who need long-term chemotherapy or patients who need long-term parenteral nutrition supplement, intravenous injection is important to them, since the medical preparations to chemotherapy and nutrition supplement are irritant to vascular endothelium and may cause phlebitis and even venous thrombosis. If these clinical scenarios occur, patients maybe need to receive repeat veno-punctures in order to establish a vascular access for intravenous injection. This is painful to patients, one from illness, the other from treatment. Consequently, an totally implantable central venous access port plays an important role in the medical field.

A prior totally implantable central venous access port catheter is a long-term vascular access and made of titanium and silicon, therefore it has higher tissue consistency in order to be implanted for a long time. The prior totally implantable central venous access port has a portal portion and it connects with a catheter with a latch. It can be totally implanted beneath the skin. The end of the catheter is placed in the junction between superior vena cava and right atrium, the portal portion is implanted within chest wall that has a membrane with the function that made for repeat puncture. A specific made needle (has been commercialized currently) may be utilized to penetrate the skin and the diaphragm of the portal portion while in usage. The totally implantable central venous access port is totally different than the external catheter and not necessary to be taken care of every day.

There are three vascular options for implanted port, including the subclavian vein, internal jugular vein, and cephalic vein. Mostly, a cut-down procedure can be done by surgeon in an operating room. The first step is to inject local anesthesia just beneath the surface of a skin. After adequate local anesthesia and vascular access confirmation, a silicon catheter is implanted via fore-mention vessels and advanced to junction between superior vena cava and right atrium under fluoroscopy. Besides, the tail portion of catheter is connected to the portal portion of port. Finally, a portal portion can be implanted beneath to skin of chest wall. Because of it, a patient may not worry about that there is no vascular access for intravenous injection. The injected medication can be directly delivered to the great vessel so as to avoid the risks that are vascular sclerosis and skin ulcer caused by the medications.

With references to FIG. 7 to FIG. 9, which illustrate a schematic sectional view of a totally implantable central venous access port in prior arts, a schematic sectional view of a path of a metal wire of the totally implantable central venous access port in prior arts and a schematic sectional view of the metal wire of the totally implantable central venous access port in prior arts. Nowadays, the totally implantable central venous access port 60 is not an ideal type yet, since a broken catheter 62 may cause complications. Once happening, the totally implantable central venous access port in patient may be removed or adjusted, so that discomfort of the patient is increased and continuity of treatment is interrupted as well. In the marketing, the totally implantable central venous access port 60 is consisted of three parts, which are a port 61, a catheter 62 and a cap (not shown in the figures). The port 61 has an injection chamber 611. The catheter 62 is female-connected to the port 61 and the cap is wedged to the body of the totally implantable central venous access port 60 after construction, therefore the connection of the cap and the body is very fragile, and a broken catheter 62 possibly happens. Further, another type that there is no connection and is integrated as one member is presented in marketing. Such type only has an injection diaphragm 612, and the only injection route is via injection diaphragm that on the top of the port 61 to injection chamber 611. A metal wire 70 may turn an angle of 90 degrees to be inserted into the catheter 62. It is a problem for the metal wire 70 being hardly implanted into the catheter 62. More, the prior metal wire 70 must be aimed at the open of the end of a puncture sheath for going through, that is not convenient for procedures. For the cases of a small blood vessel and a large angle of a blood vessel, aforesaid problem still does exist. So that how to figure out the problem of the totally implantable central venous access port 60 becomes an important issue for people skilled in the art.

### SUMMARY OF THE INVENTION

The primary objective of the present invention is to provide an improved totally implantable central venous access port, which comprises a body and a catheter, wherein the body has an injection room, a strengthened injection top diaphragm and a strengthened injection side diaphragm, the catheter is connected to one side of the body, the strengthened injection side diaphragm is at another side of the body according to the catheter, the body and the catheter are integrated as one member.

The totally implantable central venous access port further has a metal wire, which is coated a film, the length of the metal wire is 50 cm, the totally implantable central venous access port further has an puncture sheath with a side open in order to make loop around the metal wire through the puncture sheath conveniently, the metal wire has an curved tip, the strengthened injection top diaphragm and the strengthened injection side diaphragm are integrated as one member.

Comparing the present invention to the prior art, the advantages of the present invention are described as follows:
1. The body of the totally implantable central venous access port not only has the strengthened injection top diaphragm, but also the strengthened injection side diaphragm, which is at the side of the body and further has an injection hole. Since the strengthened injection side diaphragm is at another side of the body according to the catheter, the metal wire can then be effectively and conveniently inserted into the catheter. That is, the body of the prior art being not easily implanted into a patient can be solved if the blood vessel is small or the angle of the blood vessel is too sharp.
2. The present invention adopts the design of the puncture sheath with the side open. It is to conveniently and directly female-connect the metal wire, not like the prior art that an open end of the puncture sheath should be found first and aimed at, thus the metal wire can be inserted into the puncture sheath.
3. The present invention adopts the design of the body and the catheter being integrated as one member so as to reinforce the strength of the structure. Hence, the connection of the catheter and the injection base in the prior art being weak is totally solved.

Other and further features, advantages, and benefits of the invention will become apparent in the following description taken in conjunction with the following drawings. It is to be understood that the foregoing general description and following detailed description are exemplary and explanatory but are not to be restrictive of the invention. The accompanying drawings are incorporated in and constitute a part of this application and, together with the description, serve to explain the principles of the invention in general terms. Like numerals refer to like parts throughout the disclosure.

### BRIEF DESCRIPTION OF THE DRAWINGS

The objects, spirits, and advantages of the preferred embodiments of the present invention will be readily understood by the accompanying drawings and detailed descriptions, wherein:

FIG. 1 illustrates a schematic side view of a first preferred embodiment of the present invention;

FIG. 2 illustrates a schematic sectional view of the first preferred embodiment of the present invention;

FIG. 3 illustrates a schematic sectional view of a path of a metal wire of the first preferred embodiment of the present invention;

FIG. 4 illustrates a schematic sectional view of a second preferred embodiment of the present invention;

FIG. 5 illustrates a schematic sectional view of a puncture sheath of the present invention;

FIG. 5A illustrates a schematic view of an open of the puncture sheath of the present invention;

FIG. 6 illustrates a schematic view of the metal wire of the present invention;

FIG. 7 illustrates a schematic sectional view of a totally implantable central venous access port in prior arts;

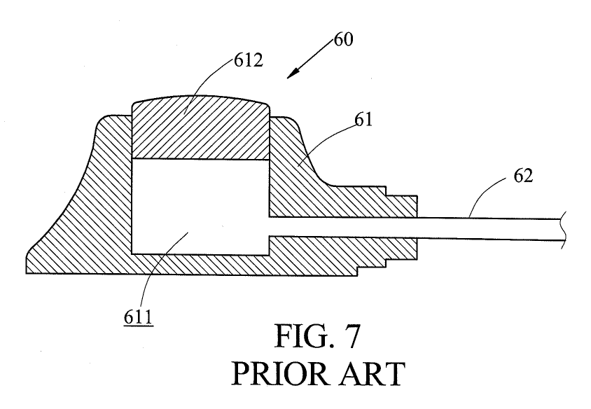

FIG. 8 illustrates a schematic sectional view of a path of a metal wire of the totally implantable central venous access port in prior arts; and

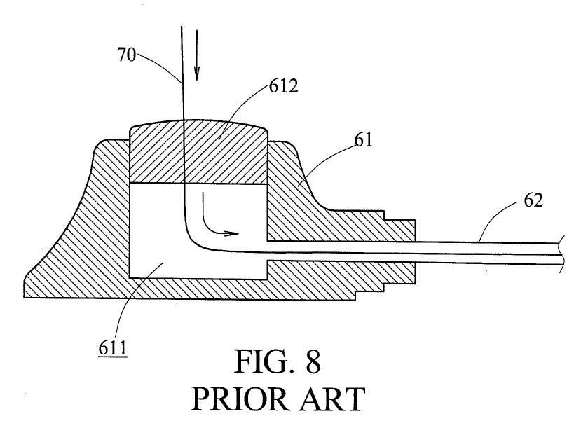

FIG. 9 illustrates a schematic sectional view of the metal wire of the totally implantable central venous access port in prior arts.

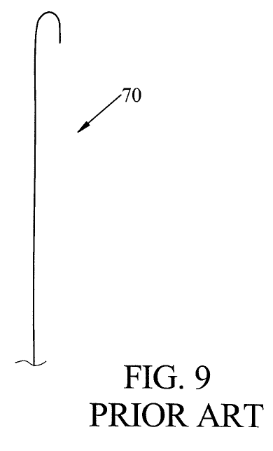

### DETAILED DESCRIPTION OF THE INVENTION

Following preferred embodiments and figures will be described in detail so as to achieve aforesaid object.

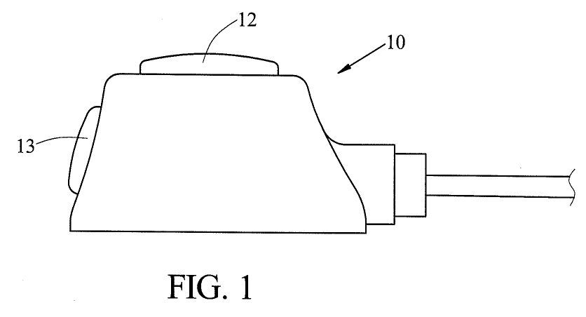

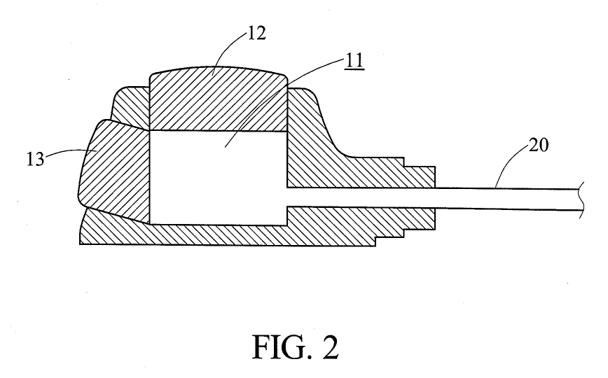

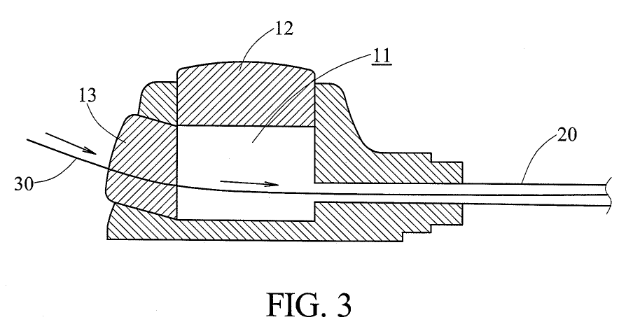

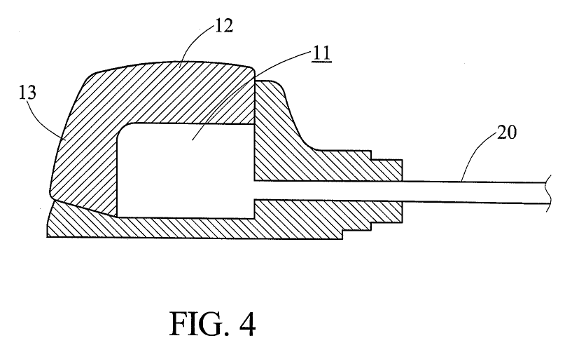

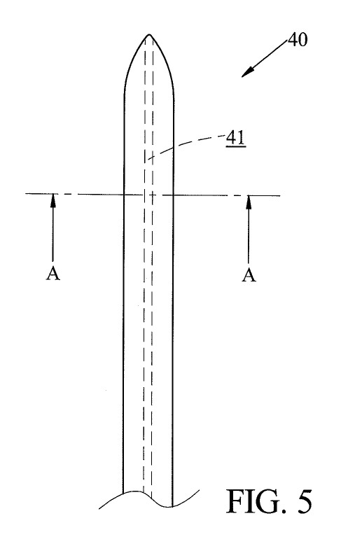

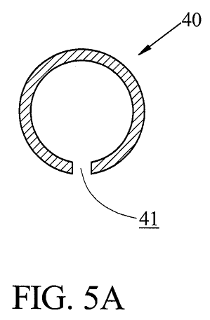

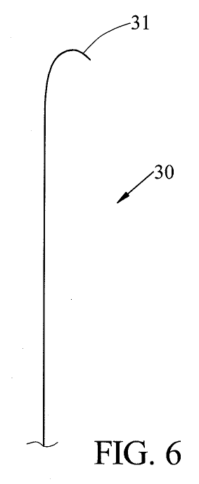

With references to FIG. 1 to FIG. 6, which illustrate a schematic side view of a first preferred embodiment of the present invention, a schematic sectional view of the first preferred embodiment of the present invention, a schematic sectional view of a path of a metal wire of the first preferred embodiment of the present invention, a schematic sectional view of a second preferred embodiment of the present invention, a schematic sectional view of an puncture sheath of the present invention, a schematic view of an open of the puncture sheath of the present invention, and a schematic view of the metal wire of the present invention. The present invention provide an improved totally implantable central venous access port, which includes a body 10 and a catheter 20, the body has an injection chamber 11, a strengthened injection top diaphragm 12 and a strengthened injection side diaphragm 13, the catheter 20 is connected to one side of the body 10, the strengthened injection side diaphragm 13 is at another side of the body 10 corresponding to the catheter 20, the body 10 and the catheter 20 are integrated as one member.

The totally implantable central venous access port further has a metal wire 30, which is coated a film, the length of the metal wire 30 is 50 cm, the totally implantable central venous access port further has an puncture sheath 40 with a side open 41 in order to make loop around the metal wire 30 through the puncture sheath conveniently, the metal wire 30 has an curved tip 31, which is slightly bended for more practical usages, but not like the J-tip of the prior art, the strengthened injection top diaphragm 12 and the strengthened injection side diaphragm 13 are integrated as one member.

Thus, the method to use the totally implantable central venous access port will be described as what follows, and the steps of the method are:
1. For the present invention, cephalic vein is the top option that the catheter 20 is implanted in, such procedure is the same as prior arts, if the blood vessels are small, the pectoralis major may then be turned over to search and make the perforator vein be through.
2. Based on the dimensions of a blood vessel, a suitable diameter and length of the catheter 20 is determined, and the procedure including the following steps:
   A. tying a knot on a far end of the blood vessel and cutting the blood vessel;
   B. implanting the catheter 20 in the blood vessel, and confirming the location of the catheter 20 through a movable X-ray machine;
   C. if the location or the curvature of the catheter 20 is not suitable, the procedure will be described as below:
   (c1) inserting a specific needle into a new injection point on a shell corresponding to a connection, and connecting the needle with the front end of the port A;
   (c2) injecting contrast to demonstrate the detail of vascular pattern and determine a path;
   (c3) inserting the metal wire 30 into the needle, and the metal wire 30 being out of the tip of the catheter 20, then building up a predetermined path for the catheter 20 through the X-ray machine;
   (c4) pushing the catheter 20 to a predetermined position; and
   (c5) drawing the metal wire 30 and the regular needle.
      Continuously, if the blood vessel is small or the angle of the blood vessel is too sharp, there have two implantation techniques, the steps of the first technique are of:
   (a1) leading the metal wire 30 to a suitable path while the blood vessel is small in a surgical operation;
   (a2) female-connecting the puncture sheath 40 to the metal wire 30;
   (a3) inserting the metal wire 30 into the puncture sheath 40;
   (a4) removing a dilator and the metal wire 30, and remaining a the puncture sheath 40 as a subcutaneous tunnel in the blood vessel;
   (a5) inputting and positioning the catheter 20 via the X-ray machine;
   (a6) implanting the body 10; and
   (a7) finishing the disposition of the body 10.
      Further, the steps of the second technique are of:
   (b1) putting the catheter 20 into the blood vessel;
   (b2) inserting the regular needle into the new injection point on the shell corresponding to the connection, and connecting the needle with the front end of the totally implantable central venous access port;
   (b3) injecting the developer to determine the path;
   (b4) inserting the metal wire 30 into the needle, and the metal wire 30 being out of the tip of the catheter 20, then building up the predetermined path for the catheter 20 through the X-ray machine;
   (b5) pushing the catheter 20 to the predetermined position along the metal wire 30;
   (b6) drawing the metal wire 30 and the regular needle; and
   (b7) finishing the disposition of the body 10.

1. (canceled)

2. A totally implantable central venous access port comprising:
a body having an injection chamber, a strengthened injection top diaphragm and a strengthened injection side diaphragm;
a catheter connected to one side of the body, with the strengthened injection side diaphragm being at another side of the body according to the catheter, with the body and the catheter being integrated as one member; and
a metal wire, which is coated with a film.

3. The totally implantable central venous access port according to claim 2, wherein a length of the metal wire is 50 cm.

4. The totally implantable central venous access port according to claim 2 further comprising a puncture sheath in the form of a tube including a wall defining a central passage extending along a length of the puncture sheath, with an opening extending along the length through the wall and open to the central passage.

5. The totally implantable central venous access port according to claim 2, wherein the metal wire has a curved tip, which is slightly bent.

6. The totally implantable central venous access port according to claim 4, wherein the strengthened injection top diaphragm and the strengthened injection side diaphragm are integrated as one member.
